(19) United States
(12) Patent Application Publication
(10) Pub. No.: US 2019/0328270 A1
(43) Pub. Date: Oct. 31, 2019

(54) MRI PATIENT SAFETY POSITIONING ASSEMBLY

(71) Applicant: Global Diagnostic Imaging Solutions, LLC, Orlando, FL (US)

(72) Inventors: Matthew Hayes, Cary, NC (US); Brian Johnson, Dallas, TX (US); Tobias Gilk, Overland Park, KS (US)

(73) Assignee: Global Diagnostic Imaging Solutions, LLC, Orlando, FL (US)

(21) Appl. No.: 15/966,928

(22) Filed: Apr. 30, 2018

(51) Int. Cl. A61B 5/055; G01R 33/30

(52) U.S. Cl. CPC A61B 5/0555; G01R 33/307

(57) ABSTRACT

A patient safety positioning assembly for MRI includes a bumper barrier configured to be releasably attached to an interior of a scanner bore of the MRI machine, wherein at least a functional portion of the bumper barrier is shaped and positioned corresponding to a boundary of a spatial gradient threshold of the MRI machine.

### FIELD OF THE INVENTION

This invention is directed toward a patient safety positioning system, and more particularly, to a patient safety positioning system during magnetic resonance imaging (MRI).

### BACKGROUND OF THE INVENTION

MRI makes use of electromagnets and short bursts of electromagnetic waves that can pass through a patient's body. These waves, in the radiofrequency range, can be used to change the orientation of hydrogen atom nuclei in the patient's tissue and thus to produce a signal that depends on tissue properties. One or more magnetic coils detects the signal and transfer it to a computer, which transforms the signals received into an image of tissue. MRI requires a large static magnetic field. The strength of the field varies as a function of distance from a MRI scanner, creating a spatial gradient magnetic field. The spatial gradient of the magnetic field exerts a translational force on ferromagnetic objects that is proportional to the gradient. For the sake of safety, there are limits on the spatial gradient magnetic field for an implanted medical device.

### SUMMARY OF THE INVENTION

Based on the foregoing, it is an object of the present invention to provide patient safety positioning system and related method of use. According to an embodiment of the present invention, a patient safety positioning assembly for MRI includes a bumper barrier releasably attached to an interior of a scanner bore of the MRI machine, wherein at least a functional portion of the bumper barrier is shaped and positioned corresponding to a boundary of a spatial gradient threshold of the MRI machine.

According to another embodiment of the present invention, a patient safety positioning assembly for MRI includes a bumper barrier configured to be releasably attached to an interior of a scanner bore of the MRI machine. At least a functional portion of the bumper barrier is shaped and positioned corresponding to a boundary of a spatial gradient threshold of the MRI machine, and a support portion of the bumper barrier is configured to be releasably attached to an end of the MRI scanner bore.

According to another embodiment, a method of controlling a patient of accessing spatial gradient zone of a MRI scanner using a patient safety positioning assembly includes attaching a support portion of the patient safety positioning assembly to an end of a MRI scanner bore, and positioning a functional portion of the patient safety positioning assembly at interior of MRI scanner bore, wherein the functional portion is shaped and positioned corresponding to a boundary of a spatial gradient threshold of the MRI machine.

### BRIEF DESCRIPTION OF THE DRAWINGS

For a fuller understanding of the invention, reference is made to the following detailed description, taken in connection with the accompanying drawings illustrating various embodiments of the present invention, in which:

FIG. 1A is a top view of an exemplary spatial gradient map of a MRI scanner;

FIG. 1B is a side cross-sectional view of an exemplary spatial gradient map of a MRI scanner;

FIGS. 2A-2C illustrate a front view, a side view, and a top view of a plurality of bumper barriers positioned to exclude access of a 5 T/m spatial gradient area;

FIGS. 3A-3C illustrate a front view, a side view, and a top view of a plurality of bumper barriers positioned to exclude access of a 7 T/m spatial gradient area;

FIGS. 4A-4C illustrate a front view, a side view, and a top view of a plurality of bumper barriers positioned to exclude access of a 10 T/m spatial gradient area;

FIG. 5 illustrates an example bumper barrier attached to a scanner of a MRI machine;

FIG. 6A illustrates a side view of a cross section of a bumper barrier in a closed position;

FIG. 6B illustrates a side view of a cross section of a bumper barrier in an open position;

FIG. 7 illustrates an example bumper barrier attached to a scanner of a MRI machine; and

FIG. 8 is a flowchart illustrating a method of controlling a patient to access spatial gradient zone of a MRI scanner using a patient safety positioning assembly.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

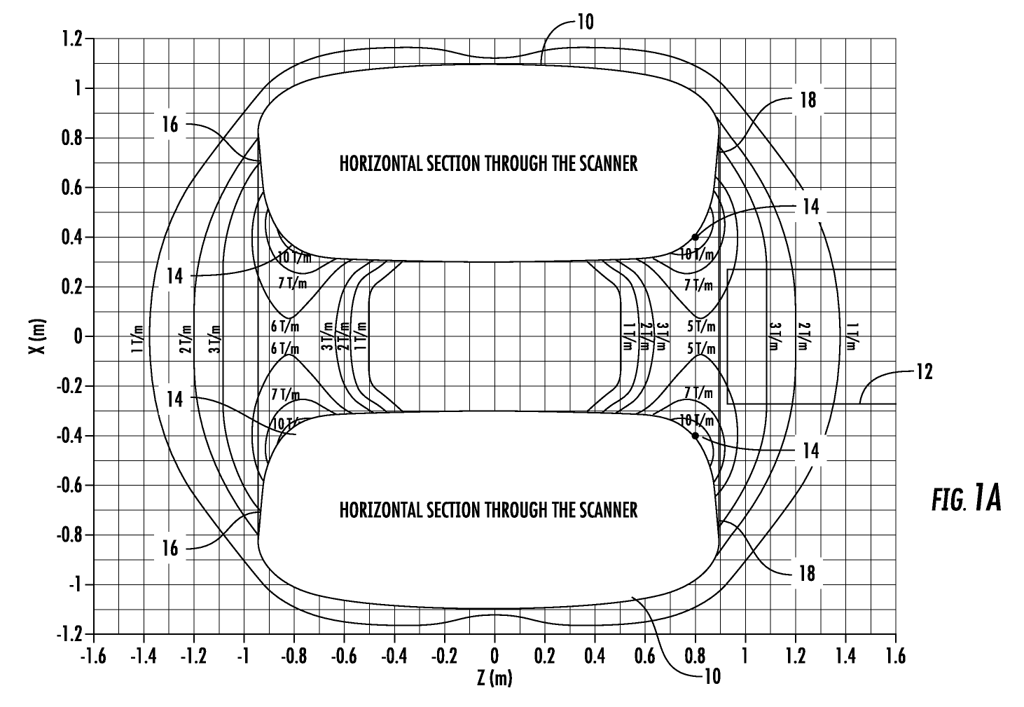

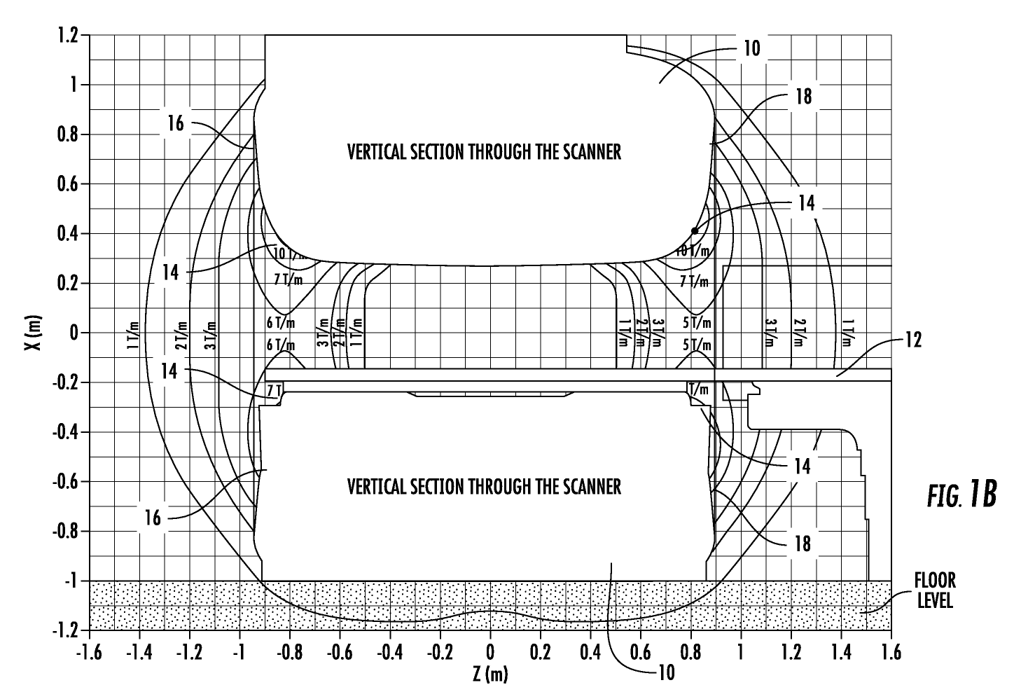

FIGS. 1 and 2 are top and side cross section view of a spatial gradient map of a 3T MRI scanner 10 and patient table 12. Gradient lines indicate different spatial gradient strengths such as 1 T/m, 2 T/m, 3 T/m, 5 T/m, 6 T/m, 7 T/m and 10 T/m, 17 T/m. The location with highest spatial gradient strength includes cover portions 14 of the front end 16 and rear end 18 of the scanner 10. The spatial gradient strength can be as high as 17 T/m. A patient safety positioning assembly such as one or more bumper barriers (not shown) can be positioned and/or sized accordingly such that a specific volume inside the scanner 12 above a specific spatial gradient value is excluded of patient access. For example, bumpers of certain dimension can be attached to certain location (e.g., a cover portion) of the scanner 10 to exclude patient access of 3 T/m, 5 T/m, and 7 T/m spatial gradient areas.

According to one embodiment of the present invention, a patient safety positioning assembly for MRI includes a bumper barrier releasably attached to an interior of a scanner bore of the MRI machine, wherein at least a functional portion of the bumper barrier is shaped and positioned corresponding to a boundary of a spatial gradient threshold of the MRI machine.

FIGS. 2A-2C illustrate a front view, a side view, and a top view of a plurality of bumper barriers 20 dimensioned and positioned to exclude patient access of 5 T/m spatial gradient area. In this case, the bumper barriers 20 are dimensioned and positioned corresponding to boundaries of a 5 T/m spatial gradient of a MRI machine. The bumper barriers 20 can be attached to the interior of the scanner bore via a hook and loop fastener such as Velcro or other suitable types of fasteners.

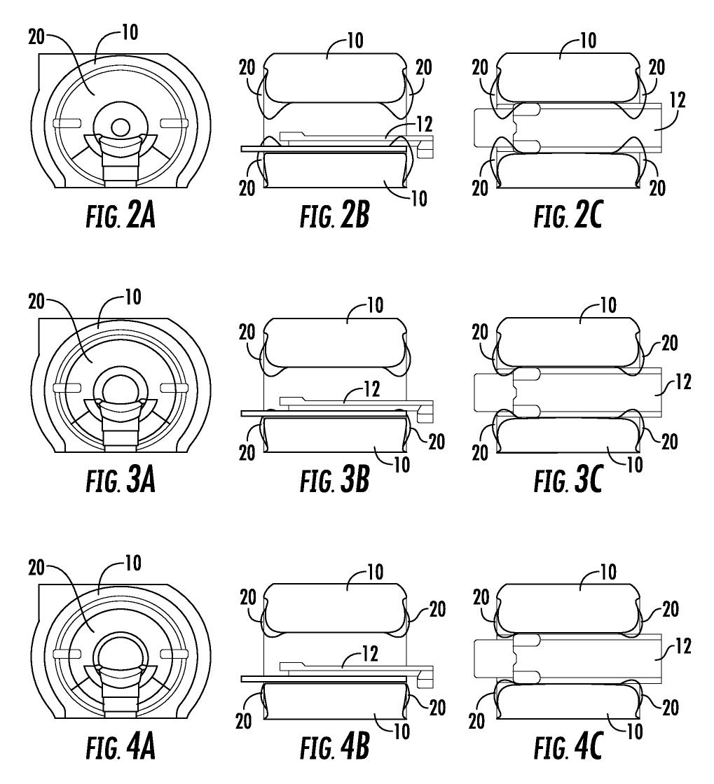

FIGS. 3A-3C illustrate a front view, a side view, and a top view of a plurality of bumper barriers 20 dimensioned and positioned to exclude patient access of 7 T/m spatial gradient area. In this case, the bumper barriers 20 are dimensioned and positioned corresponding to boundaries of a corresponding 7 T/m spatial gradient of a MRI machine.

FIGS. 4A-4C illustrate a front view, a side view, and a top view of a plurality of bumper barriers 20 dimensioned and positioned to exclude patient access of 9 T/m spatial gradient area. In this case, the bumper barriers 20 are dimensioned and positioned corresponding to boundaries of a corresponding 10 T/m spatial gradient of MRI machine.

FIG. 5 illustrates an example bumper barrier 20 attached to a scanner 10 of a MRI machine. In the depicted embodiment, the bumper barrier 20 includes an support portion 22 configured to be mounted on a front end 16 and/or rear end 18 of the scanner 10, for example, via a hook and loop fastener 26 or other suitable mounting methods. The support portion 22 is depicted as a circular flange. The bumper barrier 20 also includes a functional portion 24 connected to the circular flange 22 and dimensioned to fit inside a central bore of the MRI scanner 10. The functional portion 24 is configured to occupy certain area of the interior of the scanner 10 that has a gradient strength higher than a specific value such that a patient cannot access the area.

In the depicted embodiment, the circular flange 22 is and mounted on the rear end 18 of the scanner 10 while the functional bumper 24 is inserted in the central bore of the MRI scanner 10. The flared flange 22 can be slightly larger than the MRI bore opening. The circular flange 22 is configured to be mounted from either the front or rear of the MRI bore via a hook and loop fastener 26 or other suitable mounting methods.

According to one embodiment of the present invention, the bumper barrier 20 extends along the axis of the scanner 10 and conforms to the inner dimensions of a particular MRI scanner, as shown in FIG. 5. Alternatively, the bumper barrier is dimensioned to occupy a cover portion of the scanner 10, as shown in FIGS. 2A-4C.

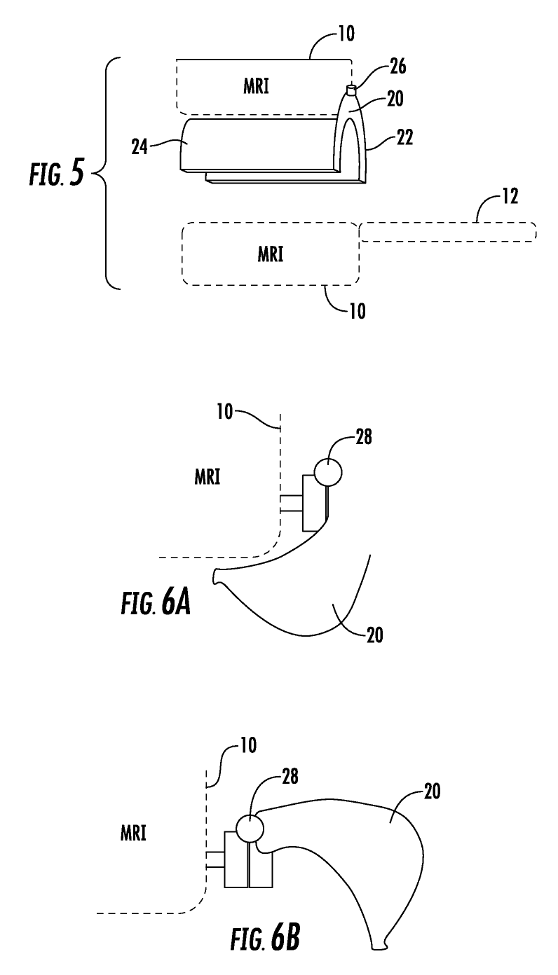

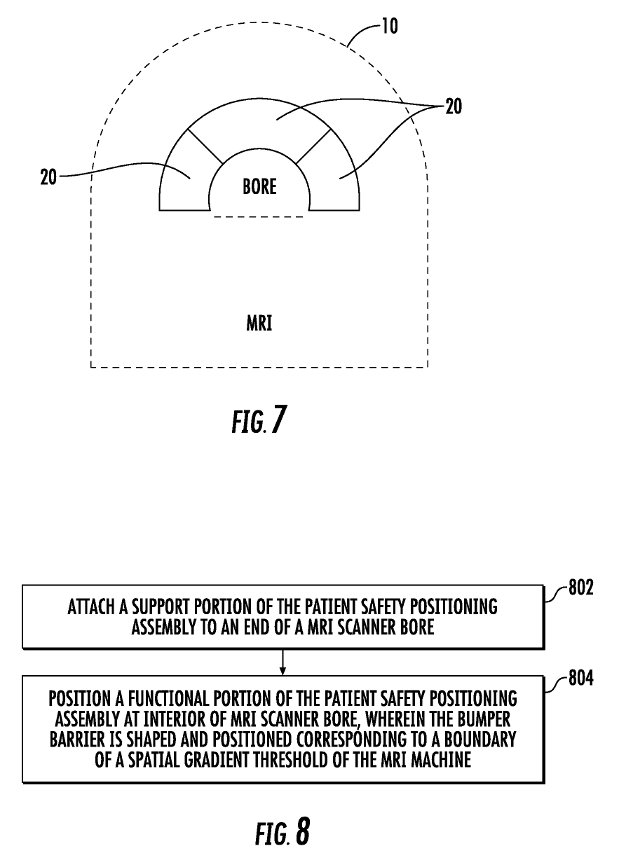

The position of bumper barriers 20 can be adjusted. FIGS. 6A and 6B illustrates a side view of a cross section of a bumper barrier 20 in a closed position and open position respectively. FIG. 6A illustrates the bumper barrier 20 in a closed position (flush with the MRI scanner 10) and configured to block certain spatial gradient zone in the scanner 10. FIG. 6B shows that the bumper barrier 20 is an open position to unblock certain spatial gradient zone in the scanner 10. The open position can accommodate for larger patients and/or patient without any implant devices. The position of the bumper barriers 20 can be adjusted between an open position and a closed position via a joint 28 (e.g., hinge joint) mounted on the scanner 10.

The bumper barrier 20 can be a single, unitary piece. Alternatively, the bumper barrier 20 can include a plurality of discrete pieces forming a unitary shape. FIG. 7 shows three discrete pieces forming a bumper barrier 20. This will make the handling and storage of bumper barrier 20 easier and faster. The discrete pieces of the bumper barrier 20 can also be configured to be in open and closed position separately.

The bumper barrier 20 is made of materials that are non-metallic, non-magnetic, and of low electrical conductivity such as rubber, foam, polyurethane, and other material with good radiolucent capabilities. The bumper barriers 20 can be cleaned by standard hospital cleaning procedure. The bumper barrier 20 is preferably made of anti-microbial material and moisture impervious.

Referring to FIG. 8, a method of controlling a patient of accessing spatial gradient zone of a MRI scanner using a patient safety positioning assembly includes at step 802, attaching a support portion (e.g., support portion 22) of the patient safety positioning assembly to an end of a MRI scanner bore. For example, the support portion is attached to an end of the MRI scanner (e.g., scanner 10) via one or more hook and loop fasteners (e.g., hook and loop fasteners 26). Other suitable fasteners can also be used.

At step 804, a functional portion (e.g., functional portion 24) of the bumper barrier is positioned in the interior of the MRI scanner bore, and the bumper barrier is shaped and positioned corresponding to a boundary of a spatial gradient threshold of the MRI machine. The position of the functional portion of the bumper barrier can be adjusted between an open position and a closed position, for example, via one or more joints (e.g., joint 28) mounted on a housing of the MRI scanner. For example, the joint can be a hinge joint, a pivot joint, a ball and socket joint, and the like. The support portion of the patient safety positioning assembly can be removed from the end of the MRI scanner when scanning is completed.

The MRI patient safety positioning assembly can prevent a specific portion of a patient with a medical device/implant from being exposed to an unsafe spatial gradient magnetic field. A safety positioning assembly can limit patient access to within a certain distance of a MRI coil during a MRI scan and prevent unsafe scanning for patients having certain implants. The safety positioning assembly can prevent patients and health care workers from entering a region with a spatial magnetic field gradient above a certain threshold. The invention will be especially useful for patients or health care workers having an implant device for which exposure to a certain spatial magnetic field gradient would be dangerous.

The safety positioning assembly is MRI compatible, safe and rapid setting, which will decrease the time to set up a patient for scanning, thereby further improving MRI productivity.

The assembly can be used in any modern non-invasive body scanning equipment based on NMR, MRI, and other techniques using large-scale superconducting magnets.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within.

What is claimed is:

1. A patient safety positioning assembly for MRI comprising:
a bumper barrier configured to be releasably attached to an interior of a scanner bore of the MRI scanner, wherein at least a functional portion of the bumper barrier is shaped and positioned corresponding to a boundary of a spatial gradient threshold of the MRI scanner.

2. The assembly of claim 1, wherein the safety positioning assembly further includes a support portion configured to be releasably attached to an end of the MRI scanner bore.

3. The assembly of claim 2, wherein the support portion is circular.

4. The assembly of claim 2, wherein the support portion is attached to an end of the MRI scanner via one or more hook and loop fasteners.

5. The assembly of claim 4, wherein the end of the scanner includes a rear end of the scanner.

6. The assembly of claim 4, wherein the end of the scanner includes a front end of the scanner.

7. The assembly of claim 1, wherein the bumper barrier is made of rubber.

8. The assembly of claim 1, wherein the bumper barrier is made of foam material.

9. The assembly of claim 1, wherein the position of the bumper barrier is adjustable between an open position and a closed position.

10. The assembly of claim 9, wherein the closed position is configured to block a space in the scanner bore having certain spatial gradient value of the MRI machine, whereas the open position is configured to unblock the space having certain spatial gradient value.

11. The assembly of claim 1, wherein the bumper barrier extends continuously along an axis of the scanner bore.

12. The assembly of claim 1, wherein the bumper barrier is located at cover portion of the scanner bore.

13. The assembly of claim 1, wherein the bumper barrier is a unitary piece.

14. The assembly of claim 1, wherein the bumper barrier includes a plurality of discrete pieces.

15. A patient safety positioning assembly for MRI comprising:
a bumper barrier configured to be releasably attached to an interior of a scanner bore of the MRI machine, the bumper barrier having a functional portion and a support portion, wherein the functional portion of the bumper barrier is shaped and positioned corresponding to a boundary of a spatial gradient threshold of the MRI machine, and the support portion of the bumper barrier is configured to be releasably attached to an end of the MRI scanner bore.

16. A method of controlling a patient of accessing spatial gradient zone of a MRI scanner using a patient safety positioning assembly, the method comprising:
attaching a support portion of the patient safety positioning assembly to an end of a MRI scanner bore; and
positioning a functional portion of the patient safety positioning assembly at interior of MRI scanner bore, wherein the functional portion is shaped and positioned corresponding to a boundary of a spatial gradient threshold of the MRI machine.

17. The method of claim 16, further comprising adjusting position of the functional portion between an open position and a close position.

18. The method of claim 17, wherein the position of the functional portion is adjusted via one or more joints mounted on a housing of the MRI scanner.

19. The method of claim 16, further comprising removing the support portion of the patient safety positioning assembly from the end of the MRI scanner when a MRI scanning is over.

20. The method of claim 16, wherein the support portion is attached to an end of the MRI scanner via one or more hook and loop fasteners.
